(19) United States
(12) Patent Application Publication
(10) Pub. No.: US 2007/0280985 A1
(43) Pub. Date: Dec. 6, 2007

(54) Methods of Establishing Intercellular Communication in an Endothelial Cell Layer and Uses Thereof

(76) Inventors: Natacha Depaola, Albany, NY (US); Denise Polacek, Philadelphia, PA (US)

Correspondence Address: ELMORE PATENT LAW GROUP, PC, 209 MAIN STREET, N. CHELMSFORD, MA, 01863, US

(21) Appl. No.: 10/574,388

(22) PCT Filed: Oct. 4, 2004
(86) PCT No.: PCT/US04/32577

§ 371 Date: May 25, 2007

Related U.S. Application Data
(60) Provisional application No. 60/508,660, filed on Oct. 3, 2003.

(51) Int. Cl. A61F 2/06; C12N 5/08; C12P 21/00

(52) U.S. Cl. 424/423; 424/93.7; 435/371; 435/70.1

(57) ABSTRACT

The present invention provides methods for generating endothelial cells in vitro, with functional intercellular communication (e.g. GJIC) that is consistent with normal endothelial cells and cell layers in vivo. In accordance with the invention, expression and appropriate organization of gap junctional proteins in endothelial cells is achieved through a) biophysical engineering, b) genetic engineering, or c) a combination of both biophysical engineering and genetic engineering. The fully functioning endothelial cells generated in vitro, in accordance with the invention are suitable for use in conjunction with substrates and matrices commonly used in tissue engineering of vascular implants.

### BACKGROUND OF THE INVENTION

One of the greatest challenges in the engineering of vascular grafts is to be able to reproduce the sophisticated functions of a natural endothelial layer. To that end, efforts of the last decade have been focused on including the use of endothelial cells in the engineering of vascular grafts. These approaches involve the use of endothelial cells that have been harvested from natural tissue, followed by the culture/amplification, and seeding of these cells on natural matrices or polymeric substrates. Unfortunately, the success of this approach has been modest. It has been reported that upon implantation and restoration of physiological flow and pressure conditions, the seeded cells are lost from the surface of the graft. Once removed from their natural environment endothelial cells are known to loose some of their functional properties. Knowing the importance of endothelial intercellular communication in maintaining vessel function it becomes a must to restore communication to cells on a vascular construct prior to implantation or use cells of functional level of communication in the engineering of these vascular constructs.

Endothelial intercellular communication occurs through gap junctions, connexin protein channels that allow the direct cell-cell transfer of ions and small signaling molecules. Endothelial Gap Junctional Intercellular Communication (GJIC) plays an important role in vascular tissue homeostasis including the coordination of cell growth and migration, vascular vasomotor responses, and angiogenesis (Ross, R., 1995, Ann Rev Physiol; 57: 791-804, Chaytor A. T, et al., 1998, J. Physiol 508, 2:561-573).

Gap junctions establish homotypic (endothelial/endothelial) and heterotypic (endothelial/smooth muscle, endothelial/circulating leukocyte) communication pathways that are essential for the maintenace of normal vascular function. Gap junctions are important in coordinating endothelial cell migration and replication during wound repair after denudation and during angiogenesis and in the propagation of signals up and down the length of the vessel to regulate blood flow through endothelium-mediated vasoregulation. The importance and extreme efficiency of GJIC between endothelial cells in vivo is well established (Segal S. S., and Beny, J. L. Am J Physiol, 1992).

Vascular gap junctions are formed by the connexin (Cx) proteins Cx37, Cx40 and Cx43. These proteins are very dynamic exhibiting rapid turnover times and variable expression patterns. Different channel properties are associated with Cx type. In humans, gap junctional intercellular communication (GJIC) is mediated primarily by Cx40 with lower expression of Cx43 and Cx37. The ability to regulate Cx43 expression with hemodynamics (fluid sheer stress, pressure) in animal cells was previously demonstrated (DePaola, N., et. al. (1999). Proc. Natl. Acad. Sci., 96:3154-3159). Other studies have also demonstrated that hemodynamics alters gap junctional intercellular communication (GJIC) and Cx43 protein expression in in vivo animal models (Gabriels, J. E. and Paul, D. L. (1998). Circ. Res., 83:636-643). However, the regulation of vascular gap junction protein expression by hemodynamics in human cells to achieve levels of protein expression, communication and distribution that resemble functional conditions in natural tissue in vitro, has not previously been demonstrated. Furthermore, there are also no prior reports of genetically engineering human endothelial cells for vascular protein (Cx) expression to generate functionally competent cells and cell layers in vitro.

It would be advantageous to provide competent endothelial cells and cell layers having fully functioning physiological gap junctional intercellular communication (GJIC), for use in the preparation of vascular grafts and other endovascular implants and devices.

### SUMMARY OF THE INVENTION

The present invention provides methods for generating endothelial cells, in vitro, with functional intercellular communication (e.g. GJIC) that is consistent with normal endothelial cells and cell layers in vivo. In accordance with the invention, the method to generate functional communication in otherwise communication-impaired cells comprises inducing the expression and organization of vascular gap junctional proteins, preferably the connexin (Cx) proteins, Cx37, Cx40 and Cx43, in endothelial cells. In accordance with the invention, expression and appropriate organization of gap junctional proteins in endothelial cells is achieved through, a) biophysical engineering, b) genetic engineering, or c) a combination of both biophysical engineering and genetic engineering. The fully functioning endothelial cells generated in vitro, in accordance with the invention are suitable for use in conjunction with substrates and matrices commonly used in tissue engineering of vascular implants and are superior to endothelial cells generated using the prior art culture procedures. The invention further provides vascular implants comprising a fully functioning endothelial cell layer produced in accordance with the methods of the invention. The invention further provides sheets of fully functioning endothelial cells that may be used in conjunction with substrates and matrices used in the manufacturing of new vascular implants, in the restoration of GJIC in existing implants, or in any indication where an implantable matrix for generating or restoring GJIC is required. The invention also provides in vivo methods of generating or restoring GJIC on an implant.

### BRIEF DESCRIPTION OF THE DRAWINGS

FIG. 1 shows the extent of cell communication is shown in histogram form, summarizing the effects on Lucifer Yellow dye transfer in HAEC exposed to 11 dynes/cm² pure laminar flow for 5-hr, 16-hr, and 24-hr and demonstrating flow exposure increases functional GJIC in a time-dependent manner. Percent (%) dye-coupling is compared to dye transfer in no-flow controls, where low activity HAEC communication ranges as 0<cells<10 and the range of communication in high activity HAEC is 2<cells<40. After 5 hours, flow exposure resulted in 1.8-fold upregulation of dye transfer. Further 3.5-fold upregulation occurred after 16 hours of flow and after 24 hours, dye transfer reached a significant a 7.5-fold increase.

FIG. 2 is a bar graph showing the results of inhibition studies using mimetic peptides to block Cx channels. In 5-hour flow conditioned samples, post-flow incubation with connexin-mimetic peptides did not affect dye-coupling when Cx43 channels were blocked. Dye-coupling was reduced by 33% only when the Cx40 channels were blocked. No reduction Lucifer Yellow dye-coupling of HAEC was observed when the Cx43 and Cx37 channels were blocked concurrently. After 16 hours of flow, there was no decrease in dye-coupling in endothelial cells in which the Cx43 channels were blocked. Nevertheless, dye-coupling was statistically significantly reduced by 57% when Cx40 channels were blocked. Simultaneous blocking of the Cx37 and Cx43 channels decreased the flow-induced dye-coupling resulted in 26% attributed to Cx37 since Cx40 inhibition did not result in a reduction in cell-cell communication.

FIG. 3 is a densitometry analysis showing flow regulated changes to Cx40 expression. “F” represents sheared 12 dynes/cm², “W” Whole cell lysates, “U” is a slow migrating upper band, and “L” represents a lower band. Densitometry analysis was done following 5 and 16 hours of flow exposure.

FIG. 4 is a densitometry analysis showing flow regulated changes to Cx43 expression. “F” represents sheared 12 dynes/cm², “W” Whole cell lysates, “U” is a slow migrating upper band, and “L” represents a lower band. Densitometry analysis was done following 5 and 16 hours of flow exposure.

FIG. 5 is a densitometry analysis showing flow regulated changes to Cx37 expression. “F” represents sheared 12 dynes/cm², “W” Whole cell lysates, “U” is a slow migrating upper band, and “L” represents a lower band. Densitometry analysis was done following 5 and 16 hours of flow exposure.

FIG. 6 is a bar graph showing flow regulation of cell communication in wound repair. Monolayer was wounded and exposed to flow for 24 hours. Individual cells located within and far away from the wounded region were injected with 5% Lucifer Yellow (LY) and 1% tetramethylrhodamine dextran. Functional gap junctional intercellular communication (GJIC) was evaluated by counting the number of dye-coupled cells after single cell injections. Control: static no flow monolayer, Laminar: laminar flow, 10 dynes/cm2. Subscript W stands for cells in the wound region, and F for cells far from it. For reference, cells injected in a control monlayer (no flow, no wound) passed LY 5.8+−cells.

FIG. 7 is a flow chart showing in vitro high throughput experiments using Human Aortic Endothelial Cells (HAEC) directly comparing disturbed (DF) and undisturbed (UF) flow regions.

### DETAILED DESCRIPTION OF THE INVENTION

In one aspect, the invention provides methods of establishing or restoring gap junctional intercellular communication (GJIC) in an endothelial cell layer, in vitro, comprising the steps of modulating the expression, organization, and assembly of at least one vascular gap junction protein or a combination of vascular gap junction proteins in the endothelial cell layer. In preferred embodiments the vascular gap junction proteins are vascular connexin (Cx) proteins. In another embodiment, preferred vascular connexin proteins are Cx37 Cx40, Cx43 or any combination thereof.

The terms “endothelial cell layer” and “endothelial cell monolayer” are used interchangeably herein and mean endothelial cells derived from any source, preferably human endothelial cells, cultured by standard procedures to form a layer or monolayer in vitro. In vivo, endothelial cells make up the one-cell thick layer at the interface between flowing blood and the artery wall known as the endothelium. The endothelium is directly exposed to the hemodynamic shear stresses associated with all of the different flow characteristics found in circulation. The endothelium is highly responsive to the flow characteristics in the local environment.

The term “establishing or restoring endothelial gap junctional intercellular Communication (GJIC)” as used herein means to obtain an intercellular communication phenotype in the endothelial cell layer in vitro that resembles the intercellular communication consistent with the normal function of the endothelium in vivo. Endothelium intercellular communications occurs through gap junctions, made up of vascular gap junction protein channels that allow direct cell to cell transfer of ions and small signaling molecules.

Vascular gap junction proteins include those of the connexin (Cx) protein family. The connexin family of proteins in the cardiovascular system includes Cx37, Cx 40, and Cx43, collectively referred to herein as the “Cx proteins”. Of particular use in the methods of the invention are Cx37, Cx 40 and Cx43. These proteins are very dynamic exhibiting rapid turnover times and variable expression patterns. Different channel proteins are associated with Cx type. In humans, it is thought that GJIC is mediated primarily by Cx40 with lower expression of Cx43 and Cx37 (Bruzzone et al., Mol. Biol. Of the Cell, 1993; 4:7-20). However, it is well established that in vitro, Cx43 is the most prominent and perhaps the only connexin expressed (Pepper M S, Am J. Physiol. 1992; 262:C1246-1257). The present inventors have found that cultured human aortic endothelial cells exhibit an strikingly low level of functional communication (i.e. GJIC) despite the abundant expression of Cx43 at cell-cell appositions. Upon induced expression of Cx proteins other than Cx43 in accordance with the methods of the invention, cultured endothelial cells achieve functional levels of intercellular communication characteristic of endothelial cells in vivo. Therefore, without being limited to any theory, it appears that successful establishment of GJIC in endothelial cell layers in vitro is related to the appropriate levels of expression, organization and assembly of a combination of Cx proteins, and particularly Cx37, Cx40 and Cx43.

Modulating the appropriate expression, organization and assembly of vascular gap proteins in an in vitro endothelial cell layer in accordance with the invention is accomplished by biophysical manipulation of the endothelial cell layer, genetic engineering of the endothelial cell layer, or a combination of both.

As used herein “biophysical manipulation” includes subjecting the endothelial cell layer to hemodynamic forces sufficient to induce the expression, organization and assembly of at least one vascular gap junction protein or a combination of vascular gap junction proteins suitable for establishing GJIC in the endothelial cell layer. “Hemodynamic forces or “hemodynamics” as those terms are used herein are those forces that result from blood flow, and the term “flow” is also used herein to describe physiologically relevant hemodyamic forces useful in the methods of the invention. Hemodynamic forces are known to influence blood vessel structure and pathology. The vascular cells lining all blood vessels are endothelial cells, which are important sensors and transducers of the two major hemodynamic forces to which they are exposed: wall shear stress (“WSS”), which is the fluid frictional force per unit of surface area, and hoop stress, which is driven by the circumferential strain (“CS”) of pressure changes. Wall shear stress acts along the blood vessel's longitudinal axis. Circumferential strain is associated with the deformation of the elastic artery wall (i.e., changes in the diameter of the vessel) in response to the pulse of vascular pressure. Wave reflections in the circulation and the inertial effects of blood flow cause a phase difference, the stress phase angle (“SPA”), between CS and WSS. The SPA varies significantly throughout the circulation.

Hemodynamic forces are created and studied in vitro using systems capable of simulating hemodynamics. Such systems are known to those skilled in the art and are also described in the patent literature. The in vitro flow chamber model for simulating hemodyamics of disturbed and undisturbed blood flow used in the present studies is described in detail in DePaola, N., et. al. (1999). Proc. Natl. Acad. Sci., 96:3154-3159 incorporated herein by reference. In vitro, nominal flow characteristics are defined by the geometry of the experimental system (e.g., flow tube, parallel plate, cone and plate, etc.). The average wall shear stress and shear stress gradient values can be accurately estimated or directly measured (Dewey et al., 1981, J. Biomech. Eng. 103:177-188; Davies et al., 1986, Proc. Nat. Acad. Sci. USA 83:2114-2118; Olesen et al., 1988, Nature 331:168-170; DePaola et al., 1992, Arterioscler. Thromb. 12:1254-1257). Although the flow characteristics are more complex in vivo, average shear stress values can be estimated from vessel geometry and flow rates (Zarins et al., 1983, Circ. Res. 53:502-514).

In one preferred embodiment, physiologically relevant hemodyamic forces that allow for the precise regulation and control of the desired properties of the endothelial cell layer including the expression of Cx proteins and the establishment of GJIC, are achieved by controlling flow magnitude and time of exposure to flow. In one embodiment preferred flow magnitude is any flow that is greater than 0 dynes/cm², and the preferred time of exposure is dependent upon the flow magnitude. If the flow magnitude is low, a longer time period of exposure to flow is preferred whereas if the flow magnitude is higher, the exposure to flow may be shorter to achieve expression of Cx proteins and establishment of GJIC in the endothelial cell layer. For example, when an average physiological arterial mean shear stress of 10-12 dynes/cm² is used, Cx protein expression and GJIC is increased within 5 hrs and reaches a significantly functional level by 16 hrs.

The present inventors have found that GJIC is critical to endothelial cell coordinated adaptation and that endothelial cell adhesion to its substrate is one of those cellular functions dependent on functional GJIC particularly during early stages of endothelial layer remodeling and adaptation to a physiological flow environment. (DePaola, N., et al., “Endothelial Monolayer Remodeling in Disturbed Flow Fields”. Annals of Biomedical Engineering, 2000; 28:S-69; DePaola, N., et al., “Spatial and Temporal Regulation of Gap Junction Connexin 43 in Vascular Endothelial Cells Exposed to Controlled Disturbed Flows In Vitro”. Proceedings of the National Academy of Science, 96(6): 3154-3159, 1999; DePaola, N., et al. “Vascular Endothelium Responds to Fluid Shear Stress Gradients”. Arteriosclerosis and Thrombosis 12: 1254-1257, 1992.). Therefore, the lack of proper cell communication that the inventors have demonstrated in cultured human endothelial cells may explain why endothelial cells seeded on vascular graft material have consistently come off the substrate upon restoration of physiological flow conditions (constructs implanted in animal models) once removed from their natural environment. The inventor's studies have demonstrated that intercellular gap junctional communication in Human Aortic Endothelial Cells is severely impaired once cells are removed from the natural vessel and set in in vitro culture conditions. DePaola, et al. “Functional Compartmentalization of Vascular Endothelium in Disturbed Flow”. Proceeddings of the International Bio-Fluid Mechanics Symposium and Workshop, Pasadena, Calif., December 2003; Ebong, E. E, Kim, S., and DePaola, N. Flow Regulation of Gap Junctional Intercellular Coupling and Connexin Expression in Human Aortic Endothelial Cells. Abstracts of the Biomedical Engineering Society Annual Meeting, Nashville, Tenn. October, 2003. However, the present invention is based on the inventor's discovery that cultured Human Aortic Endothelial Cells (HAEC) are able to reach functional levels of communication comparable with that of natural tissue by regaining expresssion of Cx40 and Cx37. This functional level was achieved by exposing the cultured cells to controlled fluid flows of physiologically relevant characteristics such as fluid sheer stress and time of flow exposure.

The studies described herein demonstrate a dynamic regulation of vascular connexins by simulated flow, in human aortic endothelial cells. Flow increases functional gap junctional communication by de novo expression of Cx40 protein and its assembly in functional channels. Inhibition studies revealed a contribution of Cx37, but to a lesser extent. The role of Cx43 in flow-induced communication is negligible. This is consistent with what is observed in vivo in normal tissue. The inventors investigated three fluid shear magnitudes: 0, 2.5, and 12 dynes/cm² and three time periods for flow exposure: 6, 16, and 24 hours. Results from functional intercellular communication evaluated by dye injection (Lucifer Yellow, MW 476) demonstrated that cell-cell communication is significantly increased with flow exposure (1,8-, 3.5-, and 7.5-fold increased with 5, 16 and 24 hours in flow, respectively) Data was obtained from 16 independent experiments, 7 individual cell injections were performed in each monolayer studied.

The evaluation of protein expression by Western blot analysis demonstrated that control monolayers are abundant in Cx43 with low expression of Cx40 and Cx37. Upon exposure to flow, Cx40 protein expression is significantly increased.

Cx37 is also increased. Cx43 is moderately regulated regulated in total amount but showed significant alteration in protein phosphorilation. Protein Localization studies by Immunocytochemistry using specific antibodies for Cx40 (Chemicon) and Cx43 (Zymed, Chemicon), revealed that control (no-flow) monolayers are abundant in Cx43 localized at cell borders while Cx 40 and Cx 37 are scarce/rare. After 24 hrs of flow Cx40 immunofluorescence increases and it is found at cell-cell appositions. Cx 37 is also increased and found at cell appositions but to a lesser extent. Cx43 is observed/found in intracellular compartments along with decreased immunoreactivity at cell borders.

The evaluation of the specificity of functional channels using connexin-mimetic peptides to block Cx43, Cx40, and Cx43 and Cx37 simultaneously (Sigma, Genosys) was performed by treating the monolayers with the connexin-mimetic peptide inhibitors followed by dye injection to evaluate extent of cell-cell communication. Results demonstrated that 16-hr of flow exposure increased dye transfer 3.5-fold compared to no-flow controls. Post-flow incubation with connexin43-mimetic peptide did not affect dye-coupling, while Cx40 peptide indicated significant decrease (57%) of dye-coupling. Simultaneous blocking of Cx37 and Cx43 channels decreased flow-induced dye-coupling by only 26%, which is attributed to Cx37 since Cx43 inhibition did not result in a reduction of cell communication.

In yet another aspect of the invention, modulation of the appropriate expression, organization and assembly of vascular gap proteins in an in vitro endothelial cell layer in is accomplished by genetic manipulation of vascular gap junction protein expression in the endothelial cell layer. In accordance with this aspect of the invention a method of establishing or restoring GJIC in an endothelial cell layer in vitro is provided comprising the steps of: a) providing an endothelial cell layer comprising recombinant endothelial cells capable of expressing at least one vascular gap junction protein or a combination of vascular gap junction proteins suitable for establishing GJIC in the endothelial cell layer; and b) inducing expression of the vascular gap junction protein or combination of vascular gap junction proteins for a period of time suitable for establishing GJIC in the endothelial cell layer. In a preferred embodiment the vascular junction proteins are Cx37 Cx40, Cx43 or any combination thereof. As used herein, the term “recombinant cell” is meant a cell comprising a nucleic acid not normally associated with the cell (e.g. a transformed, transduced or transfected cell with a construct encoding a specific protein e.g. a connexin protein). As used herein “inducing expression of a protein” means to expose the recombinant cell to conditions that facilitate the production of the recombinant protein including standard cell culture conditions that maintain growth and viability of the cultured cells. In one example, if expression of the recombinant protein is linked to an inducible promoter, protein expression may be induced by providing those conditions that induce the promoter to drive expression of the protein. Inducing expression of the protein in recombinant endothelial cells also includes exposing the protein to physiologically relevant hemodynamic forces as will be described later.

In accordance with this embodiment, the invention uses nucleic acid compositions, including genomic and cDNA nucleic acid compositions, that encode biologically active vascular gap junction proteins, preferably Cx37, Cx40 and Cx43 (collectively referred to herein as the “Cx proteins”), or biologically active fragments, homologs, or analogues thereof suitable for expression in an endothelial cell.

The “amino acid sequences of isolated Cx proteins” means the polypeptides, having structural, regulatory, or biochemical functions associated with gap junctions obtained from any species, particularly mammalian, including human, rodenti (e.g., murine or rat), bovine, ovine, porcine, murine, or equine, preferably human, and may be natural, synthetic, semi-synthetic or recombinant, and is meant to include all naturally-occurring allelic variants, and is not meant to limit the amino acid sequences to the complete, native amino acid sequence associated with the respective Cx protein molecule.

A Cx protein genomic sequence of interest comprises the nucleic acid present between the initiation codon and the stop codon, with a Cx protein gene being of particular interest, including all of the introns that are normally present in a native chromosome. It may further include the 3′ and 5′ untranslated regions found in the mature mRNA. It may further include specific transcriptional and translational regulatory sequences, such as promoters, enhancers, etc., including about 10 kb, but possibly more, of flanking genomic DNA at either the 5′ or 3′ end of the transcribed region. The genomic DNA may be isolated as a large fragment of 100 kbp or more, or as a smaller fragment substantially free of flanking chromosomal sequence. In another embodiment, the Cx protein DNA is a cDNA, which lacks intronic sequences that may be found in the genomic DNA. The cDNA may be operably linked to a promoter that is normally associated with the connexin sequence (e.g., a promoter endogenous to the connexin gene) or that is heterologous to the connexin sequence (i.e., a promoter from a source other than the connexin sequence). The sequence of this 5′ region, and further 5′ upstream sequences and 3′ downstream sequences, may be utilized for promoter elements, including enhancer binding sites that provide for expression in tissues where the connexin polypeptide is normally expressed. The connexin sequence used can be based on the nucleotide sequences of any species (e.g., mammalian or non-mammalian (e.g., reptiles, amphibians, avian (e.g., chicken)), particularly mammalian, including human, rodent (e.g., murine or rat), bovine, ovine, porcine, murine, or equine, preferably rat or human) and can be isolated or produced from any source whether natural, synthetic, semi-synthetic or recombinant.

The nucleic acid compositions used in the present embodiments of the invention may encode all or a part, usually at least substantially all, of the connexin polypeptide as appropriate. Fragments may be obtained of the DNA sequence by chemically synthesizing oligonucleotides in accordance with conventional methods, by restriction enzyme digestion, by PCR amplification, etc. For the most part, DNA fragments will be of at least about 100 contiguous nucleotides, usually at least about 200 nt, more usually at least about 250 nt to about 500 nt.

The connexin genes are isolated and obtained in substantial purity, generally as other than an intact mammalian chromosome. Usually, the DNA will be obtained substantially free of other nucleic acid sequences that do not include a sequence encoding a Cx protein or fragment thereof, generally being at least about 50%, usually at least about 90% pure and are typically “recombinant”, i.e. flanked by one or more nucleotides with which it is not normally associated on a naturally occurring chromosome.

The sequence of the connexin protein, including flanking promoter regions and coding regions, may be mutated in various ways known in the art to generate targeted changes in promoter strength, sequence of the encoded protein, etc. The DNA sequence or product of such a mutation will be substantially similar to one or more of the sequences provided herein, i.e. will differ by at least one nucleotide or amino acid, respectively, and may differ by at least two, or by at least about ten or more nucleotides or amino acids. In general, the sequence changes may be additions, substitutions, insertions or deletions. Deletions may further include larger changes, such as deletions of a domain or exon. Such modified connexins sequences can be used, for example, to generate constructs for introduction into cells for the purpose of promoting production of electrochemical connections.

The encoded connexin is biologically active, e.g facilitates establishment of GJIC. Without being held to theory, the connexin protein translate to the cell membrane and form intercytoplamic channels (gap junctions). Gap junctional intercellular communication (GJIC) can be evaluated by microinjecting cells with a gap junction permeable dye, e.g., Lucifer yellow (Molecular Probes, Or.), which is transferred from one cell to another when functional gap junctions are present. A micro injection protocol for detecting functional gap junctions (i.e. functional expression of Cx43) is given in the Examples section below.

The recombinant cells can optionally be genetically modified to express other proteins (such as one or more types of Cx proteins). Constructs comprising connexin nucleic acids are well known in the art. Constructs comprising connexin-encoding nucleic acids are utilized to transform, transfect or transduce specific cells of interest to allow for the expression of an introduced connexin-encoding nucleic acid molecule in the modified cell. Where the nucleic acid to be expressed is DNA, any construct having a promoter (e.g., a promoter that is functional in a eukaryotic cell) operably linked to a DNA of interest can be used in the invention. The constructs containing the DNA sequence (or the corresponding RNA sequence) which may be used in accordance with the invention may be any expression construct suitable for use in a mammalian cell, and containing the DNA or the RNA sequence of interest. Such constructs can include nucleic acid of a plasmid or viral construct (e.g. adeno associated virus, adenovirus, and the liked) and can be circular or linear. Preferably the construct is capable of replication in eukaryotic and/or prokaryotic hosts. Suitable constructs are known in the art and are commercially available. The constructs can be prepared using techniques well known in the art. Likewise, techniques for obtaining expression of exogenous DNA or RNA sequences in a genetically altered host cell are known in the art. In one embodiment, the DNA construct contains a promoter to facilitate expression of the DNA of interest within a mammalian cell. The promoter may be a strong promoter that functions in mammalian cells, such as a promoter from cytomegalovirus (CMV), mouse mammary tumor virus (MMTV), Rous sarcoma virus (RSV), lenti-virus or adenovirus. More specifically, exemplary promoters include the promoter from the immediate early gene of human CMV (Boshart et al., Cell 41:521530, 1985) and the promoter from the long terminal repeat (LTR) of RSV (Gorman et al., Proc. Natl. Acad. Sci. USA 79:6777-6781, 1982). Alternatively, the promoter used may be a strong general eukaryotic promoter such as the actin gene promoter.

The constructs of the present embodiments of the invention may also include sequences in addition to promoters which enhance and regulate connexin expression in modified cells. In another embodiment, the promoter is a regulated promoter (e.g., inducible promoter), such as a tetracycline-regulated promoter, expression from which can be regulated by exposure to an exogenous substance (e.g., tetracycline). Another example of regulated promoter system useful in the present invention is the lac operator repressor gene regulatory system to regulate mammalian promoters.

For eukaryotic expression, the construct should contain at a minimum a eukaryotic promoter operably linked to a DNA of interest, which is in turn operably linked to a polyadenylation signal sequence. The polyadenylation signal sequence may be selected from any of a variety of polyadenylation signal sequences known in the art. An exemplary polyadenylation signal sequence is the SV40 early polyadenylation signal sequence. The construct may also include one or more introns, where appropriate, which can increase levels of expression of the DNA of interest, particularly where the DNA of interest is a cDNA (e.g., contains no introns of the naturally-occurring sequence). Any of a variety of introns known in the art may be used (e.g., the human globin intron, which is inserted in the construct at a position 5′ to the DNA of interest.

In an alternative embodiment, the nucleic acid delivered to the cell is an RNA encoding a connexin protein. In this embodiment, the RNA is adapted for expression (i.e., translation of the RNA) in a target cell. Methods for production of RNA (e.g., mRNA) encoding a protein of interest are well known in the art, and can be readily applied to the product of RNA encoding connexin useful in the present invention.

An expression construct that provides for production of a Cx protein is then introduced into the cells which may be propagated and cultured in vitro before and/or after transformation to increase the number of recombinant connexin-expressing cells available. Methods for introducing connexin constructs into a mammalian cell include standard protocols known to those skilled in the art. The regulation of connexin expression can be accomplished using regulatory elements operably inserted into the construct comprising the connexin gene used to transduce the modified cells. Other methods of regulating connexin expression may include genomic regulatory elements endogenous to the recombinant cells or by the addition of compounds that modulate connexin expression (e.g., either at the time of or following implanting the recombinant cells.)

Connexin expression in the modified cells can be detected by such techniques as western blotting, utilizing antibodies specific for the recombinant connexin. Other methods for confirming the expression of a recombinant connexin in transformed cells may involve RT-PCR utilizing primers specific for connexin mRNA or immunofluorescence techniques on transformed cells in culture. The ability of a connexin polypeptide, to facilitate the formation of functional gap junctions can be tested as described above and in the examples, using a dye assay.

In a preferred embodiment of the invention, a method for establishing or restoring GJIC in an endothelial cell layer in vitro is provided comprising the steps of: a) providing an endothelial cell layer comprising recombinant endothelial cells capable of expressing at least one vascular gap junction protein or a combination of vascular gap junction proteins suitable for establishing GJIC in the endothelial cell layer; b) exposing the endothelial cell layer to hemodynamic forces suitable for causing the expression, organization, and assembly of at least one vascular gap junction protein or a combination of vascular gap junction proteins in the endothelial cell layer; and c) continuing to expose endothelial cell layer to hemodynamic forces until GJIC is established. This method may optionally include the step of inducing expression of the vascular gap junction protein or combination of vascular gap junction proteins prior to step (b). In one preferred embodiment the vascular gap junction proteins are Cx37 Cx40, Cx43 or any combination thereof.

The inventors have already have found that the expression of vascular gap junction proteins is flow sensitive. (DePaola, N., et. al. (1999). Proc. Natl. Acad. Sci., 96:3154-3159). Therefore, expression of vascular gap junction proteins is expected to be altered with flow. The advantage of the insertion of a construct with a promoter into the endothelial cells is that expression of the desired combination of vascular gap junction proteins will occur right away. The advantage of combining the two approaches, that is, the combination of the biophysical approach of simulating hemodynamic forces on the endothelial cells, with the genetic engineering approach to Cx expression, is that other important functions are expected to be restored by the exposure of the endothelial cell layer to the physiologically relevant flow environment via adaptation to a new dynamic condition that will promote and aid integration of the engineered construct with the natural surrounding tissue after implantation of the functionalized endothelial cell layer. Communication between endothelial cells is normally at high levels in native tissue. Endothelial cells function better once they are together as one “continuous” layer. Cells that have been genetically engineered to express combinations of Cx proteins are preferably seeded in monolayers before being exposed to controlled flow stimulation for further regulation of cell communication and other key cell functions. Prolonged exposure to physiological flow (biophysical engineering approach) is known to increase intercellular adhesion and aid in the formation of a tight endothelial barrier (a key function of naturally functional endothelium). Cells genetically engineered to express suitable combinations of Cx proteins are expected to be more readily responsive to the flow stimulus making the combination of the two approaches a more efficient and effective way to manipulate the final characteristics of these genetically engineered endothelial cell monolayers lining the constructs for implantation.

In yet another aspect, the invention provides an implant, preferably a vascular implant, comprising an endothelial cell layer having established GJIC wherein the endothelial cell layer is produced by a) biophysical manipulation of vascular gap junction protein expression in the endothelial cell layer, b) genetic manipulation of vascular gap junction protein expression in the endothelial cell layer, or c) a combination of both biophysical manipulation and genetic manipulation of the endothelial cell layer all as described above. Implants such as cardiovascular constructs (e.g. vascular grafts, heart valves) and other vascular implants (e.g. stents) comprising the fully functioning endothelial cell layer having established GJIC are superior to prior art implants in that they possess antithrombotic and proactive surfaces that are only attainable with a fully functional endothelial lining.

In another aspect the invention provides an implant, preferably a vascular implant comprising a matrix with a monolayer of recombinant endothelial cells capable of expressing at least one vascular gap junction protein or a combination of vascular gap junction proteins suitable for establishing GJIC in the endothelial cell layer. Preferred vascular gap junction proteins are the Cx proteins (Cx37, Cx40, Cx43 or any combination thereof). Suitable physiologically acceptable matrices and substrates for implants include but are not limited to, a) acellular or decellularized tissues, b) non-biodegradable, natural or synthetic polymers, c) resorbable materials including biodegradable, natural or synthetic polymers.

The term “resorbable material” as utilized herein, is defined as a material which is capable of being disassembled from its original molecular form by the human body and optionally eliminated from the human body by one or more mechanisms within the human body (for example, typically within one year of implantation). Resorbable materials include but are not limited to natural and synthetic substantially resorbable polymers such as those described in the Biomedical Engineering Handbook, p. 612, (1995), which is incorporated herein by reference. Examples of these substantially resorbable polymers include polyglycolides, polydioxanones, polyhydroxyalkanoates, polylactides, alginates, collagens, chitosans, polyalkylene oxalate, polyanhydrides, poly(glycolide-co-trimethylene carbonate), polyesteramides, polydepsipeptides and the like. More specifically, substantially resorbable polymers include polyglycolic acid and polylactic acid, and copolymers of glycolic acid with lactic acid or .epsilon.-caprolactone or trimethylene carbonate. In addition to these polyesters are the polyester-ethers such as poly-p-dioxanone.

Examples of non-biodegradable synthetic polymers suitable for use in implant matrices include but are not limited to, low density polyethylene, polypropylene, polytetrafluoroethylene (PTFE), poly 2(hydroxyethylemetharcylate) poly HEMA, polyethylene tetraphalate (PET, Dacron), poly(lactide-co-glycolide), poly dimethylsiloxane, poly (etherurethane urea), and knitted double velour polyethylene.

Acellular tissues suitable for use in the present invention include but are not limited to, pericardial matrix, or matrices derived from other tissues such as SynerGraft® (Cryolife Kennesaw, GA) derived from bovine ureter, submucosal collagen from small instestine, pleural matrix and the like. In another embodiment, the invention provides an endothelial monolayer sheet comprising an endothelial cell monolayer having established GJIC wherein the endothelial cell monolayer is produced as a result of biophysical engineering, genetic engineering or a combination thereof as discussed above. As used herein, the term “sheet”, means a matrix, preferably a thin matrix such as a film, capable of flexibly supporting a fully-functioning endothelial lining with established GJIC. The sheet is particularly useful for attaching to a luminal surface of an implant such as those described above, or for use as a sleeve, for example, that is capable of being inserted into a conventional stent at the time of the stenting procedure to serve as a natural interface between the stent and the circulating blood. The matrix may comprise a) acellular or decellularized tissues, b) non-biodegradable, natural or synthetic polymers, c) resorbable materials including biodegradable, natural or synthetic polymers, all as described above.

In another aspect, the invention provides a method for treating a patient in need of a vascular implant comprising: a) seeding an implant matrix with a monolayer of endothelial cells comprising recombinant endothelial cells capable of expressing at least one vascular gap junction protein or a combination of vascular gap junction proteins suitable for establishing GJIC in the endothelial cell layer; b) exposing the endothelial cell layer to hemodynamic forces suitable for causing the expression, organization, and assembly of at least one vascular gap junction protein or a combination of vascular gap junction proteins in the endothelial cell layer; c) continuing to expose endothelial cell layer to hemodynamic forces until GJIC is established; and e) placing the implant matrix in the patient. Optionally, the method may further comprise the step of inducing expression of the vascular gap junction protein or combination of vascular gap junction proteins prior to step (b).

In yet another aspect, the invention provides a method for treating a patient in need of a vascular implant comprising: a) seeding an implant matrix with cDNA encoding Cx37, Cx 40, Cx 43 or combination thereof; and b) placing the implant matrix in a patient. In this embodiment the cDNA for the Cx proteins are immobilized in a matrix to locally transfect autologus cells that migrate into the implant. This approach relies on cellular invasion from circulating blood endothelial projenitor cells and the adjacent natural tissue to achieve endothelialization of the graft in vivo.

In another aspect, the invention provides a process for manufacturing an implant, preferably a vascular implant, comprising the steps of: a) providing a physiologically acceptable implant matrix; b) seeding the implant matrix with a monolayer of endothelial cells comprising recombinant endothelial cells capable of expressing at least one vascular gap junction protein or a combination of vascular gap junction proteins suitable for establishing GJIC in the endothelial cell layer; c) exposing the endothelial cell layer to hemodynamic forces suitable for causing the expression, organization, and assembly of at least one vascular gap junction protein or a combination of vascular gap junction proteins in the endothelial cell layer; and d) continuing to expose endothelial cell layer to hemodynamic forces until GJIC is established. The method may optionally further comprise the step of inducing expression of the vascular gap junction protein or combination of vascular gap junction proteins prior to step (c).

### EXAMPLES

### Example 1

### Flow Apparatus

A parallel plate chamber was connected to a recirculating flow circuit composed of a variable-speed peristaltic pump, a fluid capacitor that damped pulsation, and a reservoir with culture medium. The flow chamber consisted of a Teflon upper plate and a stainless-steel bottom plate held together by eight screws. A medical-grade silicon gasket was used to seal the chamber and avoid fluid leakage. A precisely machined recess (1×30×120 mm) on the top plate defined the flow path in the chamber. The top plate also housed inlet and outlet ports and a quartz window for light transmission and sample visualization. The bottom plate was machined flat and polished to a mirror finish with an opening 62 mm from the flow entrance for sample placement. Uniform laminar, disturbed, and pulsatile flows can be created with the flow apparatus. Pulsatile flows are generated by superimposing a pulse to the main flow using a programmable syringe pump. Local disturbed flows, that simulate areas of the human vasculature susceptible to atherosclerosis, are generated using a rectangular step, 30-mm long, 1-mm wide, and 0.4-mm high, on the coverslip surface (located upstream from the region of interest with its largest dimension perpendicular to the flow). Such step created a localized region of flow separation and recirculation (flow disturbance) within the chamber. The step overlapped the glass coverslip by 4 mm on each side to minimize flow-edge effects. Temperature was maintained at 37°, and pH and oxygen levels were controlled with a 95% air/5% CO₂ humidified gas mixture blown over the medium in the reservoir.

### Example 2

### Determination of Flow Characteristics

Because the channel height in the flow chamber was much less than its width, the flow was considered two-dimensional. The fully developed channel flow, away from the step disturbance, had a uniform wall shear stress given by t=6 μU/H, where U is the mean velocity of the flow through the channel, H is the channel height, and μ is the dynamic viscosity of the fluid. In the disturbed flow region, the wall shear stress was nonuniform. The spatial variation in shear stress in regions of flow separation and recirculation was obtained from the numerical solution of the flow equations by using finite-element models. The computational domain was a two-dimensional straight channel with a rectangular step on one of the walls. The aspect ratio of the step (height/width) was 0.4, and the ratio of the channel gap to the step height (H/h) was 2.5. The two-dimensional steady Navier-Stokes equations were solved by using the computational program NEKTON (Maday, Y. & Patera, A. T. Noor, A. K. & Oden, J. T., eds. (1989) in State-of-the-Art Surveys on Computational Mechanics (Am. Soc. Microbiol., Washington, D.C.). The boundary conditions used to solve the equations were: (i) nonslip condition at the channel surfaces, (ii) a parabolic velocity profile (fully developed Poiseuille flow) assumed at the inflow, and (iii) at the outflow, the requirement that the same mass flow rate exited the computational domain as entered it. On the basis of these calculations, the flow velocity (U) and the medium viscosity (μ) were adjusted to produce regions of flow separation, reattachment, and flow recovery over the same endothelial monolayer at physiological levels of shear stress.

### Example 3

### Cells

Two well characterized strains of bovine aortic endothelial cells (BAEC; passages 6-8), initially isolated from yearling calf thoracic aortas, were cultured on glass coverslips that contained a fine photoetched grid (Bellco Glass), using standard techniques (Gimbrone, M. A., Jr. Spaet, T. H., ed. (1976) in Progress in Hemostasis and Thrombosis (Grune and Stratton, New York). Coverslips were coated with 0.1% gelatin, or, for in situ hybridization experiments, a gelatin/CrK(SO₄)₂ (0.5:0.05%) subbing solution was used. Monolayers were grown under static conditions in standard culture medium and transferred to the flow apparatus when confluent.

### Example 4

### Flow Experiments

Confluent endothelial monolayers were exposed to steady disturbed flows for 5, 16, and 24 h. In some studies, the viscosity of the flow medium was increased to 1.74 cP by the addition of 1 gm % dextran (Mᵣ 5×10⁵) to the standard culture medium to obtain higher wall shear stresses at relatively low fluid velocities. Previous experiments have demonstrated that dextran concentrations up to 10 gm % in culture medium are compatible with normal growth behavior and long-term viability of BAEC cultures (DePaola, N., Gimbrone, M. A., Jr., Davies, P. F., & Dewey, C. F., Jr. (1992). Arterioscler. Thromb. 12, 1254-1257, and erratum (1993) 13, 465). The fluid velocity through the channel was chosen to produce either unidirectional undisturbed laminar flows or disturbed flow fields in which flow separation, reattachment, and recovery could be observed within a 6-mm region downstream of the surface step. The wall shear stress in the undisturbed (no step 0 flow studies or in the fully developed flow downstream from the flow disturbance was 13.5 dynes/cm². In the disturbed flow experiments, the predicted length of the recirculation region was 1.32 mm, and the absolute value of the wall shear stress within the recirculation region ranged from 0 to 8.5 dynes/cm².

### Example 5

### In Situ Hybridization and Immunocytochemistry

Immediately upon completion of flow exposure, coverslips were removed, rinsed briefly in buffered PBS, fixed in 4% paraformaldehyde (20 min), rinsed in PBS, dehydrated in graded alcohols, and stored desiccated and frozen at −70° C. until processed for in situ hybridization for evaluation of Cx43 mRNA expression. cDNA was prepared from fresh rat heart total RNA, and a unique 308-bp fragment was subcloned into the RNA transcription vector Bluescript ML3+ (Stratagene) as described previously (Polacek, D., Bech, F., McKinsey, J. F., & Davies, P. F. (1997). J. Vasc. Res. 34, 19-30). Specificity of the antisense Cx43 riboprobe was demonstrated by hybridization to a single 3.0-kb Cx43-specific RNA species on Northern blots (id). Cx43 mRNA expression was visualized by using an Olympus CH30 microscope equipped with a dark-field condenser and Achromat ×20/0.40 numerical aperture objective lens.

Coverslips for immunohistochemical evaluation of Cx43 protein expression were rinsed briefly in PBS, fixed in ice-cold methanol/acetone (1:1) for 20 min, and processed immediately after fixation as described previously (id). A well characterized mouse mAb (Chemicon) directed to the C terminus of Cx43 was used. Control cultures were plated on similar surfaces at the same time as experimental slides and maintained under no-flow conditions in a standard 37° C., humidified 95% air/5% CO₂ incubator, in the same culture medium as the experimental cultures, for the time intervals indicated. Controls were fixed and processed simultaneously with corresponding flow samples.

### Example 6

### Dye Injections

Functional gap junctional intercellular communication (GJIC) was evaluated by microinjection of 5% Lucifer yellow (M r 457.3) and 1% tetramethylrhodamine dextran (Mᵣ 3,000) in 0.1 M LiCl/0.05 M Tris, pH 7.8. Using an automated injection system (Eppendorf Injectman) with a fast time constant, approximately 15 cells from the recirculation area and 15 cells from the recovered flow area were injected from each flow slide. Dye injections on each flow slide were completed within 15 min from the end of the flow run. Injections were alternated between flow-recirculation regions and recovered-flow regions on each slide to achieve time matching of dye spread from pairs of injected cells. The entire time to inject 30 cells was rapid compared with the turnover of Cx43 protein (30 sec/cell or 60 sec/alternating pair). After injections, cells were fixed in 4% PFA in PBS, which cross-linked both cells and dyes. Injections and dye-spread images were recorded on Kodak TMAX 100 film using an Olympus IMT2 fluorescence-inverted microscope.

### Example 7

### Image Analysis

Image analysis software IMAGEPRO (Media Cybernetics, Silver Spring, Md.) was used to quantify the spatial variation in cellular grain density associated with Cx43 mRNA expression. Digitized images were transferred to a Pentium 166 computer using a Hitachi 3CCD HV C20 video camera and a FlashPoint (Integral Technologies, Indianapolis, Ind.) frame grabber. Image areas (3.2×10⁻⁴ cm²) were collected at each of 20 consecutive locations (spaced 0.4 mm from each other) downstream from the edge of the step (60-140 images per sample). The average number of cells in each analyzed image was 50. To quantify Cx43 mRNA expression, matched phase-contrast and dark-field images were obtained (total of 120-280 images per sample). Ten randomly located images were chosen for analysis of no-flow control samples. Cx43 mRNA expression (number of grains per cell) at each location was quantified by evaluating the surface area occupied by the silver grains normalized by the average grain size of the sample and by the number of nuclei counted in the matching phase-contrast image.

### Example 8

### Hemodynamic Regulation of Gap Junctional Channel Gating and Multiple Expressions of Connexins in Human Endothelial Cells In Vitro

We have investigated the dynamics of gap junction regulation under flow conditions by evaluating the expression and assembly of the connexin proteins into functional gap junction structures for various flow exposure times (2, 5, 16, and 24 hrs). Working with human aortic endothelial cells (HAEC) exposed to physiologically relevant flows, we evaluated flow-mediated changes in the expression and localization of Cx40 and Cx43 (immunohystochemistry) and assessed GJIC by dye-coupled cell injections. Western blot analysis was conducted to quantify protein expression and determine the presence of phosphorylated forms of the various Connexin proteins (Cx 37, Cx40 and Cx43) in an attempt to explore flow-induced “gating” of assembled gap junction channels.

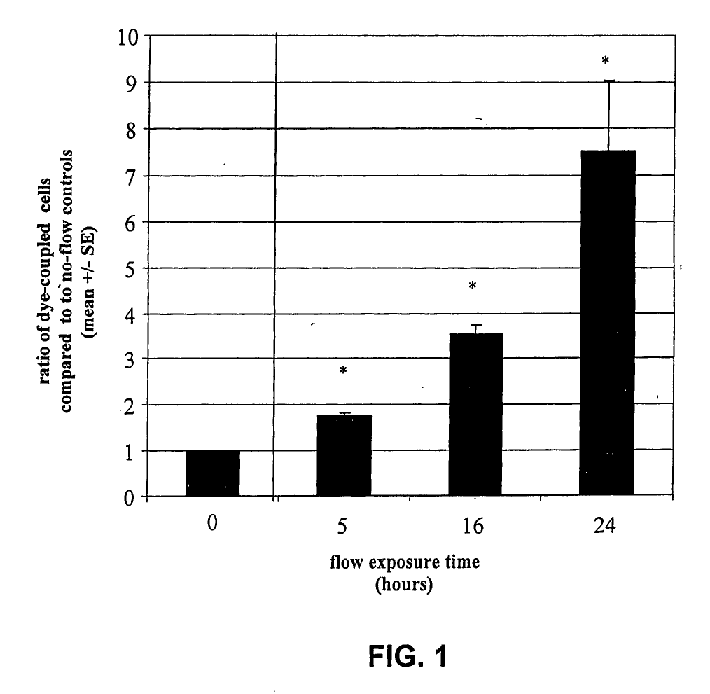

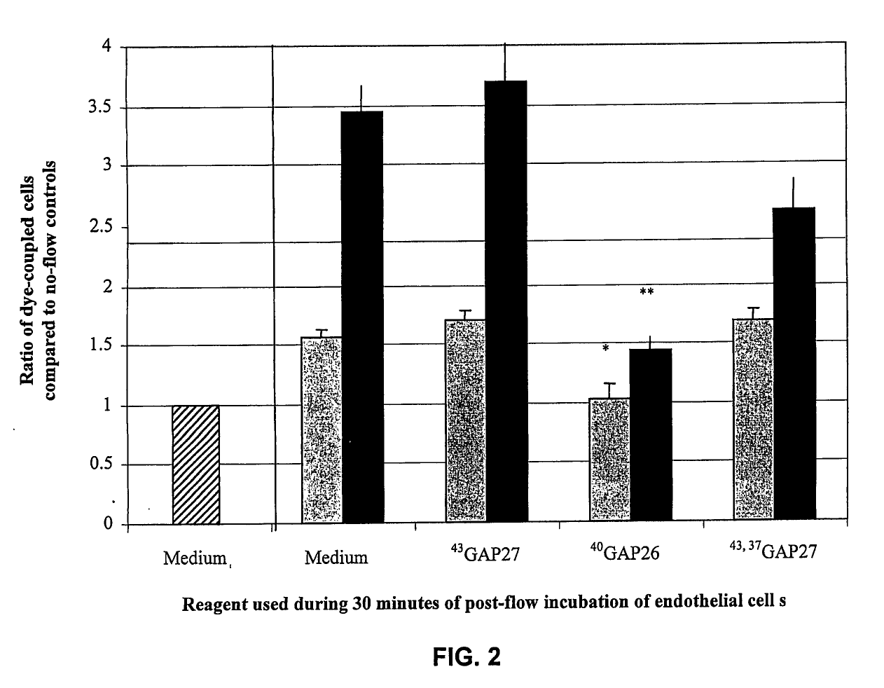

In addition, GJIC inhibition studies were conducted using Cx-mimetic peptides (which selectively block each or a combination or vascular gap junction types) to determine which of the Connexin channels were involved in the observed regulation of functional GJIC under flow conditions. Cx proteins demonstrated a very dynamic response to flow with variable expression patterns and significant alteration in functional communication, as demonstrated by dye injection. Control (no-flow) endothelial monolayers exhibited an abundant expression of Cx43 located at cell-cell apposition accompanied by a moderate expression of Cx40, as revealed by immunohistochemistry. Cx37 protein expression was not evaluated due to the unavailability of the Cx37 antibody. Dye-coupling progressively increased with time (5, 16, and 24 hrs) in HAEC exposed to physiological flows (shear stress of 11 dynes/cm²) (FIG. 1). Simultaneous blocking of Cx37 and Cx43 channels, by post-flow incubation with the Cx mimetic peptide (^{43,37}Gap27-SRPTEKTIFII (SEQ ID NO: 1)), decreased flow-induced GJIC by 25%, while blocking of Cx43 channels alone (⁴³Gap26-VCYDKSFPISHVR (SEQ ID NO: 2)) didn't alter dye-coupling (FIG. 2). The results of these inhibition studies indicate that flow-induced GJIC is mediated primarily by Cx40 with a partial contribution of Cx37 and no detectable participation of Cx43. These results are very interesting since it is well established that in vitro, Cx43 is the most prominent, and perhaps the only connexin expressed (Pepper M S. Am J Physiol. 1992; 262:C1246-1257). Nevertheless, in vivo it is thought that GJIC is mediated primarily by Cx40 (Bastide B, et al. Circ Res. 1993; 73:1138-1149, and Bruzzone, R. et al. Mol Biol of the Cell. 1993; 4:7-20) regardless of the fact that vascular endothelial cells in vivo are immunopositive for Cx40, 43, and 37.

Our latest results support the idea that with adaptation to flow, cultured endothelial cells may not only regain expression of Cx proteins other than Cx43, but that the structure and function of these newly expressed Connexins will also be similar to that of gap junctions in vivo. Inhibition studies using the mimetic peptide ⁴⁰Gap27 (SRPTEKNVFIV (SEQ ID NO: 3)) to selectively block Cx40 channels alone are currently being conducted in our laboratory.

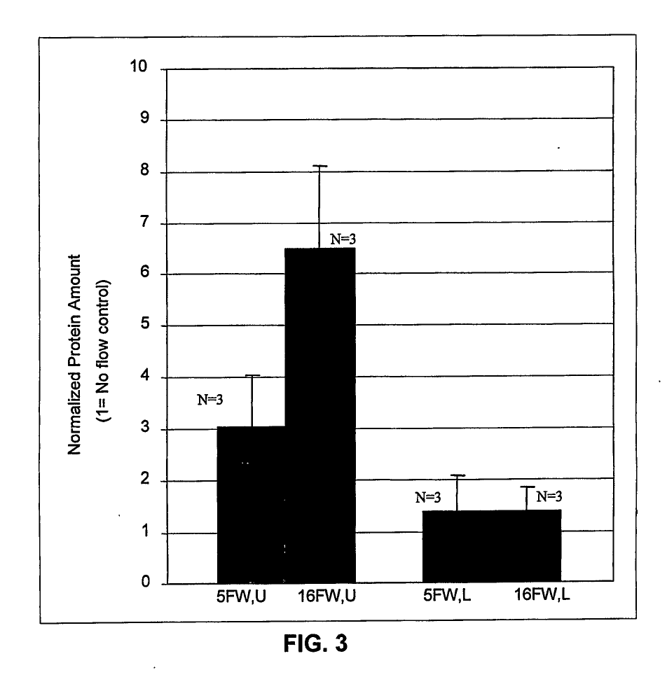

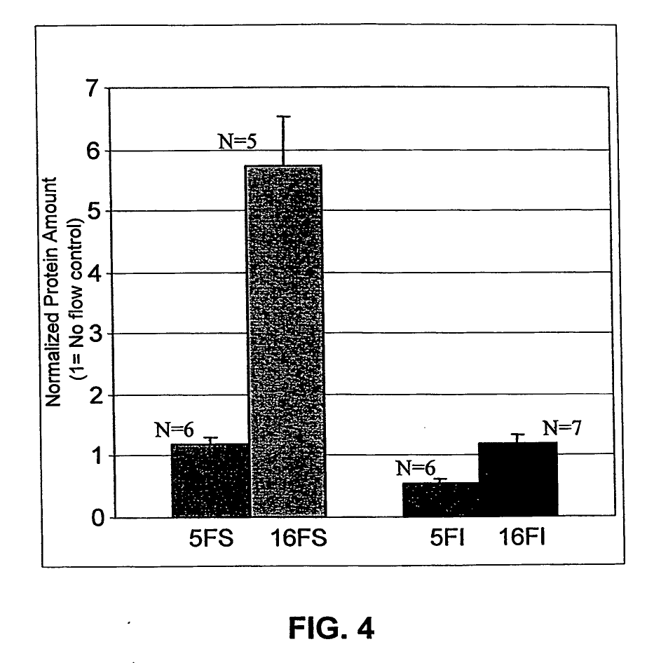

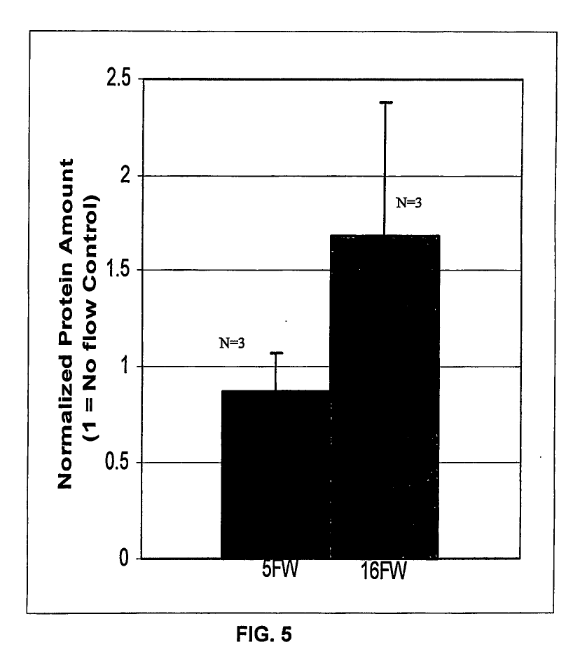

Western blot analysis and the densitometry data derived therefrom demonstrated significant changes in Cx 37, Cx43 and Cx40 protein expression under flow conditions (FIGS. 3, 4 and 5). There is a significant increase in the phosphorylated form of Cx40 with no obvious change in the non-phosphorylated form of this protein. Expression of the phosphorylated form of Cx43 remained unchanged while its non-phosphorylated form progressively increased with exposure to flow. Cx37 expression also increased. Phosphorylation of the Cx proteins has been associated with assembly of gap junctional plaques, generation of functional channels, and regulation of channel pore size and open-state probability. In these studies, alterations in functional communication and redistribution of Cx immunofluorescence were accompanied by phosphorylation of the Cx40 protein.

All together the results reported above clearly demonstrate a dynamic regulation of endothelial GJIC by fluid flow that involves switching of the functional connexin channels in a time and shear dependent fashion.

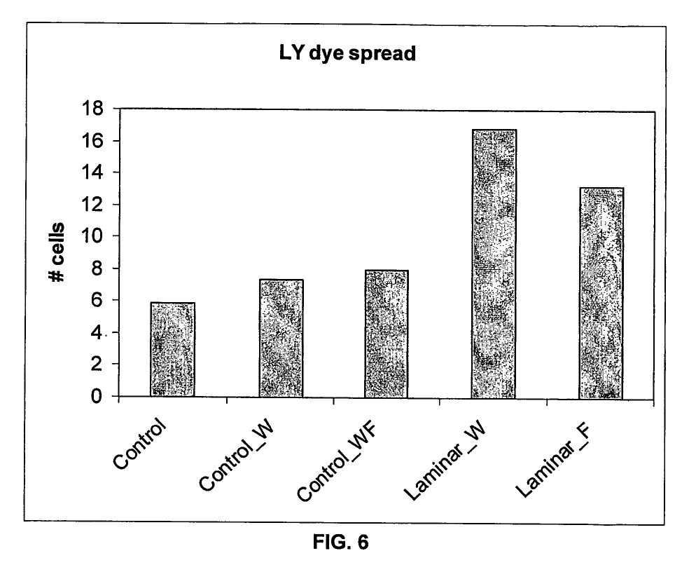

Flow regulation of GJIC during wound repair was also evaluated as part of the studies conducted this past period. It has been shown that gap junctions are important in coordinating endothelial cell migration and replication during wound repair after denudation and during angiogenesis. FIG. 6 shows the extent of cell communication evaluated by dye transfer when a wounded monolayer is allowed to repair under flow conditions (10 dynes/cm²) compared with a wounded monolayer that repairs under static (no-flow) conditions. It has been previously demonstrated that wound healing in vitro is accelerated in the presence of flow, however the role of GJIC under such conditions has not been investigated before. Our results point to flow-induced GJIC as a key component in the repair process of wounded endothelium in its natural flow environment.

To evaluate flow-mediated alterations in endothelial phenotype, a functional monocyte adhesion assay was performed on endothelial monolayers preconditioned with variable flow conditions. This study is the first report of a detailed characterization of flow-induced endothelial cell adhesiveness for leukocytes demonstrating that shear stress selectively induces the co-expression of ICAM-1 and E-selectin in a limited number of endothelial cells supporting clustered-leukocyte adhesion. This cluster pattern of adhesion (which strongly resembles in vivo leukocyte adhesion observed in early and late stage atherosclerosis) is not uniformly distributed in the monolayer as shear stress up-regulates E-selectin expression only in a subset of ICAM-1 expressing cells. Flow mediated E-selectin expression is never observed in the absence of ICAM-1. These findings support the idea of a naturally heterogeneous endothelium in which individual cells, or small group of cells, selectively respond to the local flow environment, potentially contributing to the focal origin of arterial diseases such atherosclerosis.

To analyze the expression of Cx43, 40, and 37 as well as other genes of interest in the various flow-defined regions we are using microarray analyses. Microarray analyses for high-throughput transcription profiling typically require micrograms of total RNA. Such amounts are unavailable from the limited numbers of cells use in the in vitro experiments of disturbed and undisturbed flow in this grant. In these circumstances RNA amplification is required prior to array hybridization. Although mRNA amplification is necessary for microarray analyses from limited amounts of cells and tissues, the accuracy of transcription profiles following amplification has not been well characterized. Two methods of mRNA amplification commonly in use are PCR-based and T7-driven linear amplification. While PCR is well suited to detecting gene expression in very limited amounts of starting material, its usefulness for analyzing complex transcriptional profiles is compromised by systematic biases introduced by exponential amplification. Linear amplification minimizes bias; however, uncertainty about the accuracy of differential gene expression remains because there is no reference to unamplified samples for comparison.

When sample size constraints make RNA amplification obligatory, little information is available to the investigator to assess the probability that the ranking of differentially expressed genes corresponds to reality. The fidelity of gene expression profiles generated from very small amounts of sample RNA representative of these procedures is of primary importance. We therefore tested the fidelity of differential gene expression following linear amplification by T7-mediated transcription in a well-established in vitro model of cytokine (tumor necrosis factor; TNF) stimulated human endothelial cells using filter arrays of 13,824 human cDNAs. Transcriptional profiles generated from amplified aRNA (from 100 ng total RNA, ˜1 ng mRNA) were compared to profiles generated from unamplified RNA originating from the same homogeneous pool. The results of this study are fully described in the manuscript entitled “Fidelity and enhanced sensitivity of differential transcription profiles following linear amplification of nanogram amounts of endothelial mRNA” (Polacek, et al, Physiol. Genomics 13:147-156 2003), see appendix. With this study, in addition to demonstrating fidelity in gene expression relative to unamplified samples, it is demonstrated that linear amplification results in improved sensitivity of detection, and enhances the discovery potential of high-throughput screening by microarrays. These data demonstrate the utility of RNA amplification to improve the sensitivity of detection of differential gene expression in our flow chamber studies of this grant where small numbers of cells are located in discretely defined differential flow fields.

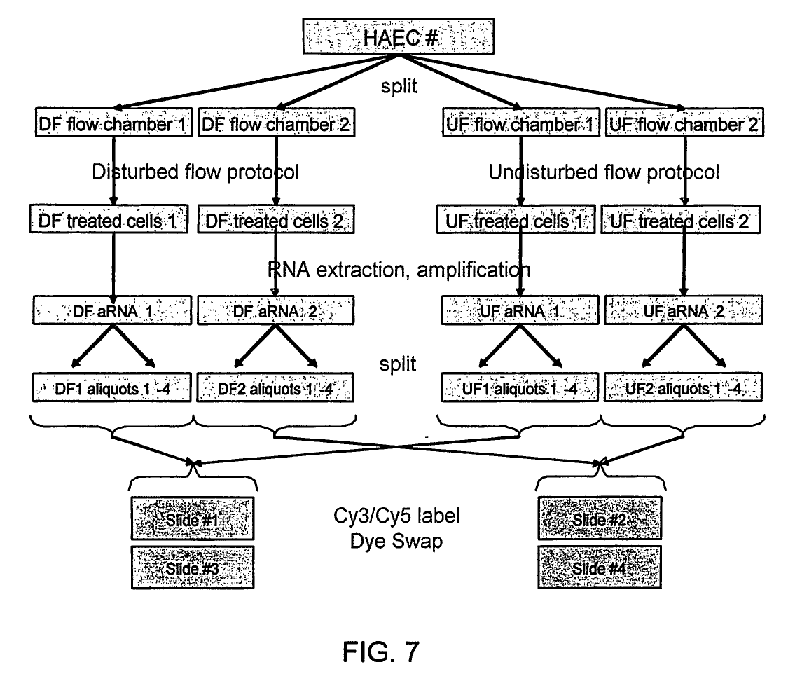

Our current in vitro high throughput experiments consists of 8 direct comparisons between Disturbed (D) and Undisturbed (U) flow regions in an in vitro model, using Agilent slides. Each such slide consists of two subarrays: A and B. Each subarray comprises spotted cDNAs (using ink-jet technology) in a layout of 60 (5×12) print tips, each printing 30×9 spots, for a total of 16200 elements. The biomaterials involved are illustrated in FIG. 7. For each of the 8 comparisons (1A, 1B, 2A, 2B, . . . , 4B), diagnostic M vs A plots (where M=log₂(D/U)) were examined, both with and without background subtraction. Only spots which were not flagged as bad were retained in the analyses (the others were assigned “NA” values). For each slide, each of the two comparisons on that slide (there were two comparisons D/U: comparison A and comparison B, performed with dye-swap on each slide) was normalized using print-tip lowess. The resulting normalized M (log₂(D/U)) values were averaged for each slide (over the two comparisons A and B) to give further normalization (gene-dye biases). This was done both with and without background subtraction. The above step yielded replicate sets of normalized M values with background subtraction and replicate sets without background subtraction. For each of the two scenarios: (i) with background subtraction, (ii) without background subtraction, the replicates were input in the stat.bayesian R-function from the sma package by the T. Speed's group (http://www.stat.berkeley.edu/users/terry/zarray/TechReport/Baypap4d.pdf) and the 16200 elements were ranked according to their lod score. The lod score represents the log of the ratio of the probability (calculated according to the algorithm described in the cited paper) that the gene is differentially expressed to the probability that it isn't, given the data and ranked accordingly. We are in the process of ranking and analyzing these data to compare disturbed with undisturbed shear stress.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of establishing or restoring gap junctional intercellular communication (GJIC) in an endothelial cell layer, in vitro, comprising the steps of modulating the expression, organization, and assembly of at least one vascular gap junction protein or a combination of vascular gap junction proteins in the endothelial cell layer.

2. The method of claim 1 wherein the vascular gap junction proteins are vascular connexin proteins.

3. The method of claim 2 wherein the vascular connexin proteins are Cx37 Cx40, Cx43 or any combination thereof.

4. The method of claim 1 wherein the modulating step is accomplished by biophysical manipulation, genetic manipulation, or a combination of both biophysical manipulation and genetic manipulation.

5. A method of establishing or restoring GJIC in an endothelial cell layer in vitro comprising the steps of:
a) providing an endothelial cell layer;
b) exposing the endothelial cell layer to hemodynamic forces sufficient to induce the expression, organization and assembly of at least one vascular gap junction protein or a combination of vascular gap junction proteins suitable for establishing GJIC in the endothelial cell layer; and
c) continuing to expose the endothelial cell layer to hemodynamic forces until the GJIC in the endothelial cell layer is established.

6. The method of claim 5 wherein the vascular junction proteins are Cx37 Cx40, Cx43 or any combination thereof.

7. A method of establishing or restoring GJIC in an endothelial cell layer in vitro comprising the steps of:
a) providing an endothelial cell layer comprising recombinant endothelial cells capable of expressing at least one vascular gap junction protein or a combination of vascular gap junction proteins suitable for establishing GJIC in the endothelial cell layer; and
b) inducing expression of the vascular gap junction protein or combination of vascular gap junction proteins for a period of time suitable for establishing GJIC in the endothelial cell layer.

8. The method of claim 7 wherein the vascular gap junction proteins are Cx37 Cx40, Cx43 or any combination thereof.

9. A method of establishing or restoring GJIC in an endothelial cell layer in vitro comprising the steps of:
a) providing an endothelial cell layer comprising recombinant endothelial cells capable of expressing at least one vascular gap junction protein or a combination of vascular gap junction proteins suitable for establishing GJIC in the endothelial cell layer;
b) exposing the endothelial cell layer to hemodynamic forces suitable for causing the expression, organization, and assembly of at least one vascular gap junction protein or a combination of vascular gap junction proteins in the endothelial cell layer; and
c) continuing to expose endothelial cell layer to hemodynamic forces until GJIC is established.

10. The method of claim 9 further comprising the step of inducing expression of the vascular gap junction protein or combination of vascular gap junction proteins prior to step (b).

11. The method of claim 9 wherein the vascular gap junction proteins are Cx37 Cx40, Cx43 or any combination thereof.

12. A vascular implant comprising an endothelial cell layer having established GJIC wherein the endothelial cell layer is produced by the method claim 5.

13. A vascular implant comprising an endothelial cell layer having established GJIC wherein the endothelial cell layer is produced by the method claim 7.

14. A vascular implant comprising an endothelial cell layer having established GJIC wherein the endothelial cell layer is produced by the method claim 9.

15. A vascular implant comprising a matrix with a monolayer of recombinant endothelial cells capable of expressing at least one vascular gap junction protein or a combination of vascular gap junction proteins suitable for establishing GJIC in the endothelial cell layer.

16. The method of claim 6 wherein the vascular gap junction proteins are Cx37, Cx40, Cx43 or any combination thereof.

17. An endothelial monolayer sheet comprising an endothelial cell monolayer having established GJIC wherein the endothelial cell monolayer is produced by the method of claim 5.

18. An endothelial monolayer sheet comprising an endothelial cell monolayer having established GJIC wherein the endothelial cell monolayer is produced by the method of claim 7.

19. An endothelial monolayer sheet comprising an endothelial cell monolayer having established GJIC wherein the endothelial cell monolayer is produced by the method of claim 9.

20. A method for treating a patient in need of a vascular implant comprising:
a) seeding an implant matrix with a monolayer of endothelial cells comprising recombinant endothelial cells capable of expressing at least one vascular gap junction protein or a combination of vascular gap junction proteins suitable for establishing GJIC in the endothelial cell layer;
b) exposing the endothelial cell layer to hemodynamic forces suitable for causing the expression, organization, and assembly of at least one vascular gap junction protein or a combination of vascular gap junction proteins in the endothelial cell layer;
c) continuing to expose endothelial cell layer to hemodynamic forces until GJIC is established; and
e) placing the implant matrix in a patient.

21. The method of claim 20 wherein the vascular implant is a stent, a shunt, a heart valve, or vascular graft.

22. A method for treating a patient in need of a vascular implant comprising:
a) seeding an implant matrix with cDNA encoding Cx37, Cx 40, Cx 43 or any combination thereof; and
b) placing the implant matrix in a patient.

23. A process for manufacturing an implant comprising the steps of:
a) providing a physiologically acceptable implant matrix;
b) seeding the implant matrix with a monolayer of endothelial cells comprising recombinant endothelial cells capable of expressing at least one vascular gap junction protein or a combination of vascular gap junction proteins suitable for establishing GJIC in the endothelial cell layer;
c) exposing the endothelial cell layer to hemodynamic forces suitable for causing the expression, organization, and assembly of at least one vascular gap junction protein or a combination of vascular gap junction proteins in the endothelial cell layer; and
d) continuing to expose endothelial cell layer to hemodynamic forces until GJIC is established.

24. The method of claim 23 further comprising the step of inducing expression of the vascular gap junction protein or combination of vascular gap junction proteins prior to step (c).

25. The method of claim 23 wherein the matrix is selected from a) acellular or decellularized tissues, b) non-biodegradable, natural or synthetic polymers, or c) resorbable materials including biodegradable, natural or synthetic polymers.

26. The method of claim 23 wherein the matrix is a polymeric material selected from, low density polyethylene, polypropylene, polytetrafluoroethylene (PTFE), poly 2(hydroxyethylemetharcylate) poly HEMA, polyethylene tetraphalate (PET, Dacron), poly(lactide-co-glycolide), poly dimethylsiloxane, poly (etherurethane urea), knitted double velour polyethylene, or combinations thereof.

27. The method of claim 23 wherein the matrix is a resorbable material selected from, polyglycolides, polydioxanones, polyhydroxyalkanoates, polylactides, alginates, collagens, chitosans, polyalkylene oxalate, polyanhydrides, poly(glycolide-co-trimethylene carbonate), polyesteramides, polydepsipeptides, or combinations thereof.

28. The method of claim 23 wherein the matrix is an acellular material selected from pericardial matrix, matrices derived bovine ureter, submucosal collagen from small intestine, or pleural matrix.
